(19) United States
(12) Patent Application Publication
(10) Pub. No.: US 2025/0171629 A1
(43) Pub. Date: May 29, 2025

(54) Mass Colored Polymer Composition For Use In Medical Technology Applications

(71) Applicant: Celanese Sales Germany GmbH, Sulzbach (DE)

(72) Inventors: Kirsten Markgraf, Weinheim (DE); Kai-Uwe Toennes, Kongernheim (DE); Thomas Seelmann, Hattersheim (DE); Bruce Mulholland, Hebron, KY (US)

(21) Appl. No.: 19/037,870

(22) Filed: Jan. 27, 2025

Related U.S. Application Data
(63) Continuation of application No. 16/401,898, filed on May 2, 2019.
(60) Provisional application No. 62/666,485, filed on May 3, 2018.

(51) Int. Cl. C08L 59/02; C08K 3/013; C08K 3/22; C08K 5/00; C08K 5/098; C08K 5/12; C08K 5/20; C08K 5/315; C08K 7/14

(52) U.S. Cl. CPC C08L 59/02; C08K 3/013; C08K 3/2279; C08K 5/0041; C08K 5/005; C08K 5/0083; C08K 5/098; C08K 5/12; C08K 5/20; C08K 5/3155; C08K 7/14; C08L 2203/02

(57) ABSTRACT

A medical grade polyoxymethylene polymer composition is disclosed that has been mass colored. The composition is particularly formulated so as to meet the standards of various governmental and safety requirements needed for use in medical applications and/or in food contact applications. In one embodiment, the polymer composition is formulated to meet the standards of USP Class VI testing and/or ISO 10993 testing.

### RELATED APPLICATIONS

The present application is a continuation of U.S. patent application Ser. No. 6/401,898, having a filing date of May 2, 2019, which is based upon and claims priority to U.S. Provisional Application Ser. No. 62/666,485, having a filing date of May 3, 2018, which are incorporated herein by reference in its entirety.

### BACKGROUND

Medical grade devices must meet high standards for safety. In particular, the materials from which the devices are made must be approved for the intended uses, passing rigorous tests to identify any leachates or extracts which may harm users of the device.

Typically, such stringent standards restrict the types of additives available for use in medical device materials, such as in polymers. Many polymeric additives, for instance, are not approved for use in medical device materials or are only approved at very small quantities. Particular problems exist when attempting to colorize polymeric materials that are intended to produce medical devices. Formulating a polymer composition to have a desired color while still remaining approved for use in medical applications has remained problematic.

For example, particular problems have been experienced in attempting to produce mass colored polyoxymethylene polymers for use in medical device applications. The polyoxymethylene polymers themselves, for instance, need to meet various governmental regulations for use in medical applications. In addition, polyoxymethylene polymers do not always readily accept or homogeneously mix with many pigments and dyes. For instance, many coloring agents can cause blooming in polyoxymethylene polymers producing polymer products having surface imperfections and/or agglomerations.

In view of the above, a need exists for a mass colored polymer composition for use in medical technology applications.

### SUMMARY

In general, the present disclosure is directed to a polymer formulation which can be used in medical technology applications. More particularly, the present disclosure is directed to a mass colored polyoxymethylene polymer composition that can be formulated to fulfill multiple governmental and safety requirements enabling use of the mass colored polymer composition to be used to make medical products and/or for molded articles for food contact. For example, in one embodiment, the mass colored polymer composition can be used to mold parts for dry powder inhalers and/or insulin pens.

In one embodiment, for instance, the present disclosure is directed to a mass colored polymer composition for medical or food container applications. The mass colored polymer composition includes a polyoxymethylene polymer having a melt volume flow rate of less than about 30 cm³/10 min when tested at 190° C. under a load of 2.16 kg. The polyoxymethylene polymer is present in the polymer composition in an amount of at least about 70% by weight, such as in an amount of at least about 80% by weight, such as in an amount of at least about 90% by weight, such as in an amount of at least about 93% by weight. The polyoxymethylene polymer is combined with at least one coloring agent. For instance, in one embodiment, the polyoxymethylene polymer may be combined with at least two coloring agents such as from about three to about ten coloring agents. The one or more coloring agents are present in the polymer composition in an amount sufficient to mass color the composition. In general, the one or more coloring agents can be present in the polymer composition in an amount less than about 15% by weight. In accordance with the present disclosure, the polyoxymethylene polymer and the at least one coloring agent are selected so that the overall polymer composition passes USP Class VI and ISO 10993 testing. The polymer composition can also be formulated to be latex free and can contain no animal byproducts. In one embodiment, the polymer composition can also be melamine free.

In one embodiment, the polyoxymethylene polymer is selected such that it contains net chloroform-soluble extractives of less than 0.5 mg/in² in accordance with 21 CFR § 177.2470 to 2480 wherein the extractives are prepared according to 21 CFR § 1.75.300(d). In addition, the polymer composition can be formulated such that the composition produces less than 0.5 mg/in² of extractable formaldehyde when tested according to 21 CFR § 177.2470 to 177.2480 using chloroform. For instance, the composition may have formaldehyde extractables of less than about 10 μg/cm², such as less than about 8 μg/cm², such as less than about 6 μg/cm², such as less than about 5 μg/cm², such as even less than about 2 μg/cm². The polymer composition can also be formulated so that it passes European regulation EC 10/2011 testing.

The one or coloring agents present in the polymer composition can comprise yellow coloring agents, blue coloring agents, red coloring agents, green coloring agents, white coloring agents, black coloring agents, or mixtures thereof. Each coloring agent present in the mass colored polymer composition may contain less than 20 ppm of an acid-soluble antimony, less than 30 ppm arsenic, less than 50 ppm lead, less than 10 ppm cadmium, less than 10 ppm cobalt, less than 10 ppm copper, less than 50 ppm nickel, less than 1 ppm selenium, less than 1 ppm mercury, and less than 100 ppm zinc. The polymer composition can be in the form of compounded pellets.

Various other additives and ingredients may be present in the polymer composition in addition to the polyoxymethylene polymer and one or more coloring agents. For instance, the polymer composition may contain an acid scavenger such as tricalcium citrate. The polymer composition may also contain an antioxidant. In one embodiment, the antioxidant comprises ethylene bis(oxyethylene) bis-(3-(5-tert-butyl-4 hydroxy-m-tolyl)-propionate). In still another embodiment, the polymer composition contains a nucleant which may be a terpolymer.

In one particular embodiment, the mass colored polymer composition may also be laser markable. For instance, the polymer composition may contain a laser marking additive. In one embodiment, the laser marking additive comprises an encapsulated antimony trioxide.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 illustrates a medical device containing a polymer prepared as disclosed herein;

FIG. 2 illustrates a medical device containing a polymer prepared as disclosed herein;

FIG. 3 illustrates a medical device containing a polymer prepared as disclosed herein; and

FIG. 4 illustrates a number of medical devices containing a polymer prepared as disclosed herein.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to a mass colored polyoxymethylene (POM) polymer composition for use in medical technology applications. Of particular advantage, some embodiments of the presently disclosed polymer composition may reduce the regulatory burden on producing products certified for biocompatibility and/or food contact. For example, a polymer article prepared as herein may, in some embodiments, be certified as biocompatible and/or safe for food contact in the form of a raw material and may subsequently convey said certifications to a finished product produced therefrom.

In some embodiments, polymers prepared as herein may meet standards such as: USP Class VI testing, ISO 10993, FDA food contact standards such as Title 21 Code of Federal Regulations (CFR)), and EU food contact standards such Regulation (EC) No. 1935/2004, 2023/2006, 10/2011 Resolution AP (89) 1, Germany BfR IX, Spain Real Decreto 847/2011, and Italy Decreto 21/3/73.

Following is a summary of some relevant standards for biocompatibility, food contact, and/or medical device use. The following summary is not complete in nature and is only intended to provide exemplary criteria to be reached by compliant materials.

USP Class VI and ISO 10993 pertain to biocompatibility standards (e.g. as relating to medical devices). United States Pharmacopeia is an organization which developed USP Class testing to help minimize public health concerns over materials (e.g. polymers). In particular, USP Class VI is the most stringent of the USP standards, requiring the material to be extracted in saline solution, alcohol saline solution, polyethylene glycol, and vegetable oil under three distinct temperature profiles: first, at 50° C. for 72 hours; second, at 70° C. for 24 hours; and third, at 121° C. for 1 hour. An extract is tested by injection into two test models (animal subjects); one model is injected intravenously or intraperitoneally, and another is injected intracutaneously. The models are observed after a series of time steps (e.g. 24, 48, and/or 72 hours) to evaluate any toxic effects, both internal (e.g. illness, death) and/or topical (e.g. rash, reaction). Strips of the material are implanted into muscle tissue of the models and any reactions are evaluated after a longer period of time (e.g. 5 to 7 days).

ISO 10993 is a broader and more comprehensive standard than USP Class IV. The FDA has identified key biocompatibility endpoints for consideration, including cytotoxicity (e.g. at 37° C. for 24-73 hours), sensitization (e.g. via guinea pig maximization and local lymph node assay according to ASTM F2148), irritation or intracutaneous reactivity, acute systemic toxicity, material-mediated pyrogenicity (e.g. per USP 34 <151>), subacute or subchronic toxicity, genotoxicity (e.g. bacterial gene mutation assay according to OECD 471 (1997), mouse lymphoma gene mutation assay according to OECD 476 (1997), chromosomal aberration assay according to OECD 473 (2014), micronucleus assay according to OECD 487 (2014), a bone marrow micronucleus assay according to OECD 474 (2014), a bone marrow chromosomal aberration assay according to OECD 475 (1997), and/or a peripheral blood micronucleus assay according to OECD 474), implantation effects, hemocompatibility (e.g. hemlysis according to ASTM F756 or equivalent, complement activation according to ELISA or ASTM F1984 methods, and thrombogencity), chronic toxicity, carcinogenicity, reproductive or developmental toxicity, and degradation. Any aspect may be investigated for limited contact (up to 24 hour duration), prolonged contact (greater than 1 day and up to 30 days duration), and permanent contact (longer than 30 days duration). The investigations should reflect the proposed end use of the item/material. For example, the testing requirements may differ among the use categories suggested by the FDA, including surface devices, contacting intact skin, mucosal membranes, or breached or compromised surfaces; external communicating devices, contacting the blood path indirectly, tissue, tissue fluids, subcutaneous spaces, bone, dentin, or circulating blood; and implant devices, contacting tissue, tissue fluids, subcutaneous spaces, bone, or blood.

Title 21 CFR deals generally with food and drugs. In particular, 21 CFR § 177.2470 and § 177.2480 address POM copolymers and homopolymers, respectively. Compliant adjuvants may be added, such as stabilizers or pigments. The POM polymers may not yield net chloroform-soluble extractives exceeding 0.5 mg/in² of food contact area and are restricted to use in temperatures not exceeding 250° F. Extractives are prepared according to the simulated use scenarios set forth in 21 CFR § 175.300(d) and repeated below:

TABLE 1
Types of food.

| Label | Type of Food |
|---|---|
| I | Nonacid (pH above 5.0), aqueous products; may contain salt or |
| | sugar or both, and including oil-in-water emulsions of low- |
| | or high-fat content. |
| II | Acidic (pH 5.0 or below), aqueous products; may contain |
| | salt or sugar or both, and including oil-in- |
| | water emulsions of low- or high-fat content. |
| III | Aqueous, acid or nonacid products containing free oil or fat; |
| | may contain salt, and including water-in-oil emulsions of |
| | low- or high-fat content. |
| IV | Dairy products and modifications: |
| IV-A | Water-in-oil emulsion, high- or low-fat. |
| IV-B | Oil-in-water emulsion, high- or low-fat. |
| V | Low moisture fats and oils. |
| VI | Beverages: |
| VI-A | Containing alcohol. |
| VI-B | Nonalcoholic. |
| VII | Bakery products. |
| VIII | Dry solids (no end test required). |

TABLE 2
Test Procedures for determining amount of extractives from resinous or
polymeric coatings using solvents simulating types of food and beverages.

| | Types of Food | Extractant | | |
|---|---|---|---|---|
| Condition of Use | (See Table 1) | Water | Heptane¹ | 8% Alcohol |
| A. High temperature heat- | I, IV-B . . . | 250° F., 2 hr | | |
| sterilized (e.g. over 212° F.) | III, IV-A, VII . . . | 250° F., 2 hr . . . | 150° F., 2 hr | |
| B. Boiling water-sterilized | II . . . | 212° F., 30 min | | |
| | III, VII . . . | 212° F., 30 min . . . | 120° F., 30 min | |
| C. Hot filled or pasteurized | II, IV-B . . . | Fill boiling, cool to 100° F. | | |
| above 150° F. | III, IV-A . . . | Fill boiling, cool to 100° F. | 120° F., 30 min | |
| | V . . . | . . . | 120° F., 30 min | |
| D. Hot filled or pasteurized | II, IV-B, VI-B . . . | 150° F., 2 hr | | |
| below 150° F. | III, IV-A . . . | 150° F., 2 hr . . . | 100° F., 30 min | |
| | V . . . | . . . | 100° F., 30 min | |
| | VI-A . . . | . . . | . . . | 150° F., 2 hr |
| E. Room temperature filled | II, IV-B, VI-B . . . | 120° F., 24 hr | | |
| and stored (no thermal | III, IV-A . . . | 120° F., 24 hr . . . | 70° F., 30 min | |
| treatment in the container) | V, VII . . . | . . . | 70° F., 30 min | |
| | VI-A . . . | . . . | . . . | 120° F., 24 hr |
| F. Refrigerated storage (no | I, II, III, IV-A, | | | |
| thermal treatment in the | IV-B, VI-B, VII . . . | 70° F., 48 hr | | |
| container) | VI-A . . . | . . . | . . . | 70° F., 48 hr |
| G. Frozen storage (no | I, II, III, IV-B, VII . . . | 70° F., 24 hr | | |
| thermal treatment in the | | | | |
| container) | | | | |
| H. Frozen storage: Ready | | | | |
| prepared foods intended to | | | | |
| be reheated in container at | | | | |
| time of use. | | | | |
| 1. Aqueous or oil | I, II, IV-B . . . | 212° F., 30 min | | |
| in water emulsion | | | | |
| of high or low fat. | | | | |
| 2. Aqueous, high | III, IV-A, VII . . . | 212° F., 30 min . . . | 120° F., 30 min | |
| or low free oil or fat. | | | | |

¹Heptane extractant not to be used on wax-lined containers. Heptane extractivity results must be divided by a factor of five in arriving at the extractivity for a food product.

21 CFR § 177.2470 and § 177.2480 detail further requirements for POM polymers. The POM polymers, with or without any adjuvants, when ground or cut into particles that pass through a U.S.A. Standard Sieve No. 6 and that are retained on a U.S.A. Standard Sieve No. 10, should yield total extractives not to exceed (i) 0.2 wt. % when extracted for 6 hours in distilled water at reflux temperature, (ii) 0.15 wt. % when extracted for 6 hours in n-heptane at reflux temperature. POM homopolymers must not yield formaldehyde in an amount exceeding 0.005 wt. %. Additionally, POM homopolymers should contain stabilizers in an amount no more than 1.9 wt. %. The minimum number average molecular weight of the copolymer is 25000, with a density between 1.39 and 1.44 g/cm3 and a melting point between 172° C. and 184° C. Approved POM copolymers may be the reaction product of trioxane and either ethylene oxide or up to 5 wt. % butanediol formal. The minimum number average molecular weight of the copolymer is 15000. Approved copolymers should contain no more than 2.0 wt. % stabilizers, no one stabilizer amount being more than 1.0 wt. %.

In general, the polyoxymethylene polymer composition and the polyoxymethylene polymer itself should be relatively resistant to formaldehyde emissions. For instance, the polymer composition can be formulated so as to produce extractable formaldehyde in an amount less than 10 μg/cm², such as less than about 8 μg/cm², such as less than about 6 μg/cm², such as less than about 5 μg/cm², such as less than about 3 μg/cm², such as even less than about 2 μg/cm². The composition can also be configured to emit less than about 6 mg/kg, such as less than about 5 mg/kg, such as less than about 4 mg/kg, such as less than about 3 mg/kg, such as even less than about 2 mg/kg of formaldehyde when tested according to Test VDA 275 after 24 hours using a plaque having a width of 2 mm.

The European regulations for polymer food contact standards are found in EC 10/2011. Similar to 21 CFR, the regulation lists a number of simulated scenarios and materials to provide test conditions mimicking the realistic worst-case scenario of the proposed use of the material, for example Tables 1 and 2 of ANNEX V detail contact times and temperatures of the test scenarios using the simulated extractants listed in Table 1 of ANNEX III.

EC 10/2011 also lists total migration limitations for a variety metals per mass of food or food simulant: Barium, 1 ppm; Cobalt, 0.05 ppm; Copper, 5 ppm; Iron, 48 ppm; Lithium, 0.6 ppm; Manganese, 0.6 ppm; and Zinc, 25 ppm. Primary aromatic amines not listed in Table 1 of ANNEX I must not be released in detectable amounts (less than 0.01 ppm).

Various EU publications have further defined restrictions on additives, such as for colorants (color additives). For instance, EC 10/2011 restricts carbon black amounts to no more than 2.5 wt. % with no more than 0.25 ppm benzo(a)pyrene and the toluene-extractable fraction not exceeding 0.1 wt. %.

For example, AP (89) 1 defines the metals and metalloids in colorants may be soluble in 0.1 M HCl in amounts no more than the following: Antimony, 0.05 wt. %; Arsenic, 0.01 wt. %; Barium, 0.01 wt. %; Cadmium, 0.01 wt. %; Chromium, 0.1 wt. %; Lead, 0.01 wt. %; Mercury, 0.005 wt. %; and selenium, 0.01 wt. %. Primary aromatic amines in the colorants soluble in 1M HCl and expressed as aniline should be present in amounts not exceeding 500 ppm. Carbon black, in particular, must not contain a toluene-extractable fraction exceeding 0.15 wt. %. Extractable polychlorinated biphenyls should not exceed 25 ppm.

Germany BfR IX defines the same metal purity limitations as AP (89) 1 and additionally requires that the colorants are required to withstand temperature ranges from approximately 150° C. to approximately 300° C. while the plastic is being processed.

Spain Real Decreto 847/2011 defines the same metal purity limitations as AP (89) 1, except specifying 0.1N HCl.

Italy Decreto 21/3/73 defines the same metal purity limitations as AP (89) 1, except specifying 0.1N HCl and further restricting arsenic to no more than 0.005 wt. %.

In some embodiments, a polymer composition is formulated to meet at least one of the above-mentioned certifications by careful attention to and selection of processing parameters and composition ingredients. For example, the preparation of the polyoxymethylene polymer and the selection of any additives (e.g. color additives and/or laser marking additives) may be uniquely manipulated to produce a final product (e.g. a material, device component, or finished device) adhering to at least one of the above-mentioned standards.

The preparation of the polyoxymethylene polymer can be carried out by polymerization of polyoxymethylene-forming monomers, such as trioxane or a mixture of trioxane and a cyclic acetal such as dioxolane in the presence of a molecular weight regulator, such as a glycol. The polyoxymethylene polymer used in the polymer composition may comprise a homopolymer or a copolymer. According to one embodiment, the polyoxymethylene is a homo- or copolymer which comprises at least 50 mol. %, such as at least 75 mol. %, such as at least 90 mol. % and such as even at least 97 mol. % of —CH₂O-repeat units.

In one embodiment, a polyoxymethylene copolymer is used. The copolymer can contain from about 0.01 mol. % to about 20 mol. % and in particular from about 0.5 mol. % to about 10 mol. % of repeat units that comprise a saturated or ethylenically unsaturated alkylene group having at least 2 carbon atoms, or a cycloalkylene group, which has sulfur atoms or oxygen atoms in the chain and may include one or more substituents selected from the group consisting of alkyl cycloalkyl, aryl, aralkyl, heteroaryl, halogen or alkoxy. In one embodiment, a cyclic ether or acetal is used that can be introduced into the copolymer via a ring-opening reaction.

Preferred cyclic ethers or acetals are those of the formula:

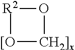

in which x is 0 or 1 and R² is a C₂-C₄-alkylene group which, if appropriate, has one or more substituents which are C₁-C₄-akyl groups, or are C₁-C₄-alkoxy groups, and/or are halogen atoms, preferably chlorine atoms. Merely by way of example, mention may be made of ethylene oxide, propylene 1,2-oxide, butylene 1,2-oxide, butylene 1,3-oxide, 1,3-dioxane, 1,3-dioxolane, and 1,3-dioxepan as cyclic ethers, and also of linear oligo- or polyformals, such as polydioxolane or polydioxepan, as comonomers. It is particularly advantageous to use copolymers composed of from 99.5 to 95 mol. % of trioxane and of from 0.01 to 5 mol. %, such as from 0.5 to 4 mol. %, of one of the above-mentioned comonomers. In one embodiment, the polyoxymethylene polymer contains relatively low amounts of comonomer. For instance, the comonomer can be present in an amount less than about 2 mol. %, such as less than about 1.5 mol. %, such as less than about 1 mol. %, such as less than about 0.8 mol. %, such as less than about 0.6 mol. %.

In one embodiment, the preparation of the polyoxymethylene can be carried out by polymerization of polyoxymethylene-forming monomers, such as trioxane or a mixture of trioxane and dioxolane, in the presence of ethylene glycol or methylal as a molecular weight regulator. The polymerization can be effected as precipitation polymerization or in the melt. Initiators which may be used are the compounds known per se, such as trifluoromethane sulfonic acid, these preferably being added as solution in ethylene glycol to the monomer. The catalyst can be a liquid, solid or gas. In one embodiment, the catalyst may comprise a boron compound, such as boron trifluroride. Boron trifluroride may be present during the processes of gas. The procedure and termination of the polymerization and working-up of the product obtained can be effected according to processes known per se. By a suitable choice of the polymerization parameters, such as duration of polymerization or amount of molecular weight regulator, the molecular weight and hence the MVR value of the resulting polymer can be adjusted.

The melting point of the polyoxymethylene polymer (or blend of polymers) can vary depending upon how the polymer is made, its molecular weight, and various other factors. In one embodiment, for instance, the melting point can be from about 150° C. to about 200° C. The weight average molecular weight of the polymer can vary from about 5000 to about 200,000, such as from about 7000 to about 150,000.

In one embodiment, the polyoxymethylene polymer used in the polymer composition may contain a relatively high amount of reactive groups or functional groups in the terminal positions. The reactive groups, for instance, may comprise —OH or —NH₂ groups.

In one embodiment, the polyoxymethylene polymer can have terminal hydroxyl groups, for example hydroxyethylene groups and/or hydroxyl side groups, in at least more than about 50% of all the terminal sites on the polymer. For instance, the polyoxymethylene polymer may have at least about 70%, such as at least about 80%, such as at least about 85% of its terminal groups be hydroxyl groups, based on the total number of terminal groups present. It should be understood that the total number of terminal groups present includes all side terminal groups.

In one embodiment, the polyoxymethylene polymer has a content of terminal hydroxyl groups of at least 15 mmol/kg, such as at least 18 mmol/kg, such as at least 20 mmol/kg. In one embodiment, the terminal hydroxyl group content ranges from 18 to 50 mmol/kg. In an alternative embodiment, the polyoxymethylene polymer may contain terminal hydroxyl groups in an amount less than 20 mmol/kg, such as less than 18 mmol/kg, such as less than 15 mmol/kg. For instance, the polyoxymethylene polymer may contain terminal hydroxyl groups in an amount from about 5 mmol/kg to about 20 mmol/kg, such as from about 5 mmol/kg to about 15 mmol/kg. For example, a polyoxymethylene polymer may be used that has a lower terminal hydroxyl group content but has a higher melt volume flow rate.

In addition to or instead of the terminal hydroxyl groups, the polyoxymethylene polymer may also have other terminal groups usual for these polymers. Examples of these are alkoxy groups, formate groups, acetate groups or aldehyde groups. According to one embodiment, the polyoxymethylene is a homo-or copolymer which comprises at least 50 mol-%, such as at least 75 mol-%, such as at least 90 mol-% and such as even at least 95 mol-% of —CH₂O-repeat units.

In one embodiment, a polyoxymethylene polymer can be produced using a cationic polymerization process followed by solution hydrolysis to remove any unstable end groups. During cationic polymerization, a glycol, such as ethylene glycol or methylal can be used as a chain terminating agent. A heteropoly acid, triflic acid or a boron compound may be used as the catalyst.

The polyoxymethylene polymer can have any suitable molecular weight. The molecular weight of the polymer, for instance, can be from about 4,000 grams per mole to about 20,000 g/mol. In other embodiments, however, the molecular weight can be well above 20,000 g/mol, such as from about 20,000 g/mol to about 100,000 g/mol.

The polyoxymethylene polymer present in the composition can generally have a melt flow index (MFI) ranging from about 0.1 to about 80 cm³/10 min, as determined according to ISO 1133 at 190° C. and 2.16 kg. In one embodiment, the polyoxymethylene polymer may have a melt flow index of less than about 30 cm³/10 min, such as less than about 25 cm³/10 min, such as less than about 20 cm³/10 min, such as less than about 15 cm³/10 min, such as less than about 10 cm³/10 min, such as less than about 5 cm³/10 min. The melt flow index is generally greater than about 0.5 cm³/10 min. In an alternative embodiment, a polyoxymethylene polymer may be used that has a relatively high melt flow index. For instance, the polyoxymethylene polymer may have a melt flow index of from about 25 cm³/10 min to about 70 cm³/10 min, such as from about 30 cm³/10 min to about 55 cm³/10 min.

Suitable commercially available polyoxymethylene polymers are available under the trade name Hostaform® (HF) by Celanese.

The polyoxymethylene polymer may be present in the polyoxymethylene polymer composition in an amount of at least 50 wt. %, such as at least 60 wt. %, such as at least 70 wt. %, such as at least 80 wt. %, such as at least 85 wt. %, such as at least 90 wt. %, such as at least 93 wt. %, such as at least 95 wt. %. In general, the polyoxymethylene polymer is present in an amount of less than about 100 wt. %, such as less than about 99 wt. %, such as less than about 97 wt. %, wherein the weight is based on the total weight of the polyoxymethylene polymer composition.

Reinforcing fibers which may be included in the composition are mineral fibers, such as glass fibers, polymer fibers, in particular organic high-modulus fibers, such as aramid fibers, metal fibers, such as steel fibers, carbon fibers, natural fibers, and/or fibers from renewable resources. The reinforcing fibers can be present in the molding composition in an amount ranging from 5 to 45 wt.-%, such as from 10 to 40 wt.-%, wherein the weight is based on the total weight of the composition. These fibers may be in modified or unmodified form, e.g. provided with a sizing, or chemically treated, in order to improve adhesion to the polymer. Glass fibers, for instance, may be used. Reinforcing fibers may be compounded into the polyoxymethylene matrix, for example in an extruder or kneader. However, the reinforcing fibers may also advantageously take the form of continuous-filament fibers sheathed or impregnated with the polyoxymethylene molding composition in a process suitable for this purpose, and then processed or wound up in the form of a continuous strand, or cut to a desired pellet length so that the fiber lengths and pellet lengths are identical. An example of a process particularly suitable for this purpose is the pultrusion process.

The polymer composition may further comprise an impact modifier such as a thermoplastic elastomer. Thermoplastic elastomers are materials with both thermoplastic and elastomeric properties. Thermoplastic elastomers include styrenic block copolymers, polyolefin blends referred to as thermoplastic olefin elastomers, elastomeric alloys, thermoplastic polyurethanes, thermoplastic copolyesters, and thermoplastic polyamides.

Thermoplastic elastomers include polyester elastomers (TPE-E), thermoplastic polyamide elastomers (TPE-A) and in particular thermoplastic polyurethane elastomers (TPE-U).

The amount of thermoplastic elastomer contained in the polymer composition can vary depending upon various factors. For instance, the thermoplastic elastomer can be present in an amount ranging from about 0.5% by weight to about 50% by weight. In one embodiment, for instance, a thermoplastic elastomer or impact modifier may be present in the composition in an amount less than about 25% by weight, such as in an amount less than about 15% by weight, such as in an amount less than about 10% by weight. The thermoplastic elastomer or impact modifier is generally present in an amount greater than about 2% by weight, such as in an amount greater than about 5% by weight, such as in an amount greater than about 8% by weight, such as in an amount greater than about 10% by weight.

In one embodiment, when an impact modifier or thermoplastic elastomer is present in the composition, the composition can also include a coupling agent. The coupling agent can be present generally in an amount from about 0.1% to about 2% by weight, such as from about 0.1% to about 1% by weight.

In one embodiment, the polymer composition may contain an acid scavenger. The acid scavenger can comprise a carboxylic acid salt. For instance, the carboxylic acid salt may comprise a salt of a fatty acid, such as a metal salt of a fatty acid. For example, the carboxylic acid salt may comprise an alkaline earth metal salt of a fatty acid. The cation of the salt, for instance, may comprise calcium, barium, lithium, sodium, magnesium, zinc, or the like.

The fatty acid can contain a carbon chain of generally from about 3 carbon atoms to about 20 carbon atoms. The fatty acid may comprise a dicarboxylic acid or a tricarboxylic acid.

In one embodiment, the metal salt of the fatty acid may comprise a metal salt of citric acid, propionic acid, stearic acid, butanoic acid, hexanoic acid, decanoic acid, lauric acid, myristic acid, palmitic acid, and the like. In one particular embodiment, the metal salt of the fatty acid may comprise calcium propionate, calcium 12-hydroxystearate, a calcium citrate such as tricalcium citrate, and mixtures thereof. In one embodiment, when the polyoxymethylene polymer composition includes one or more coloring agents, various benefits and advantages are obtained by combining the coloring agents with calcium propionate.

One or more carboxylic acid salts are generally present in the polymer composition in an amount greater than about 0.05% by weight, such as in an amount greater than about 0.1% by weight, such as in an amount greater than about 0.2% by weight, such as in an amount greater than about 0.3% by weight, such as in an amount greater than about 0.4% by weight, such as in an amount greater than about 0.5% by weight. One or more carboxylic acid salts are generally present in the polymer composition in an amount less than about 5% by weight, such as in an amount less than about 3% by weight, such as in an amount less than about 2% by weight, such as in an amount less than about 1.5% by weight, such as in an amount less than about 1% by weight.

The polymer composition of the present disclosure may also contain other known additives such as, for example, antioxidants, UV stabilizers or heat stabilizers, impact modifiers and/or reinforcing fibers. In addition, the compositions can contain processing auxiliaries, for example adhesion promoters, lubricants, nucleants, demolding agents, fillers, or antistatic agents and additives which impart a desired property to the compositions and articles or parts produced therefrom.

In one embodiment, an ultraviolet light stabilizer may be present. The ultraviolet light stabilizer may comprise a benzophenone, a benzotriazole, or a benzoate. The UV light absorber, when present, may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, a nucleant may be present. The nucleant may increase crystallinity and may comprise an oxymethylene terpolymer. In one particular embodiment, for instance, the nucleant may comprise a terpolymer of butanediol diglycidyl ether, ethylene oxide, and trioxane. The nucleant may be present in the composition in an amount of at least about 0.01 wt. %, such as at least about 0.05 wt. %, such as at least about 0.1 wt. % and less than about 2 wt. %, such as less than about 1.5 wt. %, such as less than about 1 wt. %, wherein the weight is based on the total weight of the respective polymer composition.

In one embodiment, an antioxidant, such as a sterically hindered phenol, may be present. Examples which are available commercially are pentaerythrityl tetrakis[3-(3,5-di-tert-butyl-4-hydroxyphenyl)propionate], triethylene glycol bis[3-(3-tert-butyl-4-hydroxy-5-methylphenyl) propionate], 3,3′-bis[3-(3,5-di-tert-butyl-4-hydroxyphenyl)propionohydrazide], and hexamethylene glycol bis[3-(3,5-di-tert-butyl-4-hydroxyphenyl) propionate]. In one embodiment, the antioxidant incorporated in the composition is ethylene bis(oxyethylene) bis-(3-(5-tert-butyl-4 hydroxy-m-tolyl)-propionate). The antioxidant may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, lights stabilizers, such as sterically hindered amines, may be present in addition to the ultraviolet light stabilizer. Hindered amine light stabilizers that may be used include oligomeric hindered amine compounds that are N-methylated. For instance, hindered amine light stabilizer may comprise a high molecular weight hindered amine stabilizer. Other embodiments of light stabilizers include 2,2,6,6-tetramethyl-4-piperidyl compounds, e.g., bis(2,2,6,6-tetramethyl-4-piperidyl) sebacate or the polymer of dimethyl succinate and 1-(2-hydroxyethyl)-4-hydroxy-2,2,6,6-tetramethyl-4-piperidine. In one embodiment, the light stabilizer may comprise 2-(2H-benzzotriazol-2-yl) 4,6-bis(1-ethyl-1-phenyl-ethyl) phenol. The light stabilizers, when present, may be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, lubricants may be present. The lubricant may comprise a polymer wax composition. Further, in one embodiment, a polyethylene glycol polymer (processing aid) may be present in the composition. The polyethylene glycol, for instance, may have a molecular weight of from about 1000 to about 5000, such as from about 3000 to about 4000. In one embodiment, for instance, PEG-75 may be present. In another embodiment, a fatty acid amide such as ethylene bis(stearamide) may be present. Lubricants may generally be present in the polymer composition in an amount of at least about 0.01 wt. %, such as at least about 0.05 wt. %, such as at least about 0.075 wt. % and less than about 1 wt. %, such as less than about 0.75 wt. %, such as less than about 0.5 wt. %, wherein the weight is based on the total weight of the respective polymer composition.

In one embodiment, the polyoyxmethylene polymer composition may also contain a formaldehyde scavenger. In one embodiment, the formaldehyde scavenger can be melamine free. In one embodiment, for instance, the formaldehyde scavenger may comprise a dicyandiamide alone or in combination with a copolyamide. When present, the acid scavenger can be contained in the polymer composition in an amount less than about 1% by weight, such as in an amount less than about 0.5% by weight, such as in an amount less than about 0.3% by weight, such as in an amount less than about 0.1% by weight, such as in an amount less than about 0.05% by weight and generally in an amount greater than about 0.0001% by weight.

Additionally, polymers prepared as herein may be laser markable and/or mass colored. Additionally, some embodiments may not contain latex and/or animal byproducts.

In general, laser marking polymers is one method of forming images (e.g. dots, lines, glyphs, pictures, barcodes), engravings (e.g. for visual and/or structural purposes), or other marks on a polymeric substrate. In particular, laser marking processes often offers high speed and precision, especially when compared to traditional printing processes.

Of particular advantage, laser marking does not involve the application of a second material (e.g. ink) to the surface of the polymer, minimizing the number of substances which must be certified according to various biocompatibility and food contact regulations, such as those discussed herein. Depending on the parameters of the laser marking process (e.g. laser focus point speed, temperature, wavelength, etc.), the laser marks are formed, in some embodiments, by foaming, engraving, color change and/or carbonizing. In addition to color changes, the laser marking parameters may be configured to produce marks having a greyscale shade ranging between white and black (inclusive). The penetration of any of the marking processes may be characterized by a staining depth; for example, in some embodiments a staining depth of greater than about 50 μm is desired, such as greater than about 100 μm. Generally, the staining depth is desired to be less than the thickness of the polymeric substrate to avoid compromising the mechanical characteristics of the substrate.

For example, a laser marking process may be configured to engrave (i.e. remove material) with or without changing the color of the engraved area. In some cases, the engraved area may also undergo carbonization and display a dark and clearly defined contrast between the engraved area and the surrounding polymer. In other examples, the marking process may be configured to foam a particular area to provide a distinct texture between the marked areas and unmarked areas, with or without providing a color change on the marked areas. Generally, a polymer produces a foamed area when the polymer melted by the laser produces gas bubbles which remain trapped in the cooling melt. For example, foaming may occur at low laser intensities. In some cases, foamed areas may display a lighter color than the surrounding unfoamed areas, providing a visual contrast in addition to the texture. Some embodiments may be configured to minimize the foaming height.

The equipment used for laser marking processes is available in a wide variety, following writing, mask, dot matrix, and other mode of operation. For example, a writing laser may employ at least one or more controllable mirrors to direct a laser beam to the surface of a polymeric substrate to write or otherwise mark the surface according to a pre-programmed pattern. Mask laser marking processes project a laser beam onto a masking device which only permits the passage of portions of the beam conforming to a particular pattern cut into the mask, and the portions which pass through the mask are directed to the surface of the subject polymeric substrate. Dot matrix laser systems employ a shutter device to compose markings from closely grouped but individual dot marks.

An example of traditional laser marking systems are cw or pulsed CO₂ lasers and yttrium aluminum garnet (YAG) (e.g. Nd:YAG) lasers where the marking is accomplished by the heat of the applied laser beam. The wavelengths of the pulses produced by these systems are within the visible or infrared spectrum. A pattern or indicia to be marked is formed by using a mask through which the laser beam passes or by a focused laser beam which is moved or scanned to produce the desired indicia or pattern. Such lasers are also employed for engraving, soldering and welding wherein, the case of marking, the surface layer of the material is melted, ablated or vaporized to produce discernible indicia or pattern. Also, this type of article marking may be accomplished by use of a chemical reaction at the article surface to be marked where certain coating agents on the surface of the article, which may be visibly transparent, but undergo a visible contrast change under the influence of a laser beam or laser pulses.

CO₂ lasers have been principally employed for marking plastic surfaces, such as packages. The laser beam from the laser is directed through a copper stencil to form the indicia on the plastic surface. However, due to the shrinkage of some packages over the years, CO₂ lasers, in many cases, are no longer suitable since high quality indicia with good visibility is no longer satisfactory for this particular application. However, low cost, lower marking quality CO₂ systems employing low cost X-Y galvanometer devices are still employed for applications not requiring high quality marking.

YAG lasers are extensively employed for package marking as well as many other marking applications. YAG lasers have shorter wavelengths of operation permitting the marking of indicia on harder surfaces, such as ceramic material. The beam in the YAG marking systems is steered or scanned in one, two or three dimensions by means of a pair of displaceable mirrors mounted for rotation to displace a laser beam in orthogonal directions to form a two-dimensional scan of the beam on the surface to be marked, such as, for example, a X-Y galvanometer device or a X-X galvanometer device operated under computer control. Indicia is scribed onto the surface of an article to be marked with fine resolution and marking clarity on comparatively smaller surfaces, such as in the case of smaller packages.

However, some polymers do not absorb the laser energy in the desired wavelengths (e.g. UV to infrared) and fail to produce the desired marking results. Therefore, some embodiments may employ a laser marking additive to enhance or otherwise permit the marking of plastics in IR, visible, and UV wavelength ranges. For example, even for materials which are laser markable, contrast or other marking characteristics may be improved upon incorporation of a laser marking additive.

Laser marking additives identified as meeting all requirements for incorporation to a polymer as described herein include, but are not limited to, Iriotec 8208. Iriotec 8208 is representative of compounds wherein an antimony trioxide is encapsulated within microspheres and is carried within a polymeric matrix (e.g. polyethylene). The mass ratio of matrix to antimony trioxide may range between about 35:65 to about 25:75.

A laser marking additive may be incorporated into the polymer in an amount above 0.1 wt. % in some embodiments, such as above about 0.5 wt. %, such as above about 1 wt. %, such as above about 2 wt. %. In some embodiments, the additive is present in an amount less than about 4 wt. %, such as less than about 3 wt. %, such as less than about 2 wt. %.

Advantageously, such an additive may not require any special processing parameters apart from those used in the processing of the polymer as described herein. In some cases, a temperature increase of 10° C. to 20° C. above the original temperature may be optimal. Dispersion of the additive, in some cases, may be improved when the polymer-additive mixture is processed above about 100° C., such as above about 160° C. In some embodiments, the processing temperature is kept below about 300° C., such as below about 280° C., such as below about 250° C.

Mass colored polymers are polymers which are colored throughout the material, i.e. more than just a surface coating of coloration. In some embodiments, mass coloration is achieved by mixing a pigment or other color additive into the polymer during processing. The coloring agent may be present in the composition in an amount of at least about 0.01 wt. %, such as at least about 0.05 wt. %, such as at least about 0.1 wt. %, such as at least about 0.5 wt. %, such as at least about 0.8 wt. %, such as at least about 1 wt. % and less than about 5 wt. %, such as less than about 2.5 wt. %, such as less than about 1 wt. %, wherein the weight is based on the total weight of the respective polymer composition. In some examples, the coloring agent may be present in amounts greater than about 2 wt. %, such as greater than about 4 wt. %, such as greater than about 6 wt. %, such as greater than about 8 wt. %, such as greater than about 10 wt. %. In various embodiments, the coloring agent may be present in an amount less than about 15 wt. %, such as less than about 13 wt. %, such as less than about 11 wt. %, such as less than about 9 wt. %, such as less than about 7 wt. %, such as less than about 5 wt. %.

For use in a medical device or an object certified for food contact, both the pigment and the polymer must meet high standards for safety. For example, as written in US Title 21 of the Code of Federal Regulations, the FDA requires that, absent evidence suggesting otherwise, a safety factor of 100 must be used when relying upon animal trials to demonstrate safety for human use. Typically, the FDA considers evidence regarding the oral toxicity, primary irritation, sensitization, subacute dermal toxicity on intact and abraded skin, and carcinogenicity of external color additives, although some tests may be waived upon demonstration that such tests are not required to determine safety for the proposed use.

Pigments identified as meeting all requirements for incorporation to a polymer as described herein include, but are not limited to, Sicotan Yellow K 2112, Kronos 2220, Kronos 2211, Kronos 2233, Printex FP, PV Fast Green GNX, PV Fast Yellow HG, Irgazin Yellow K 2070, Bayferrox 3910, Irgazin Red K 3840, Cromophtal Orange GP, Heliogen Blue K 7090, and Heliogen Green K 8730. Specifically disclosed pigments are to be understood as merely representative examples of the variety of pigments that may be used.

Sicotan Yellow K 2112, a rutile pigment based on chromium III oxide, antimony pentoxide, and titanium dioxide. Any acid-soluble antimony is present in an amount less than about 20 ppm. Additionally, unavoidable impurities are suppressed to 30 ppm arsenic, 50 ppm lead, less than 10 ppm cadmium, less than 10 ppm cobalt, less than 10 ppm copper, less than 50 ppm nickel, less than 1 ppm selenium, less than 1 ppm mercury, and less than 100 ppm zinc. Sicotan Yellow K 2112 complies with or otherwise is permitted by the following rules and regulations: EU regulation no 1935/2004/EC—Art. 3, AP(89)1, Germany BfR IX, and Australian regulation AS 2070 -1999, being conditionally compliant with use restrictions under EU (EC) Regulation 10/2011, France Brochure 1227, Spain Real Decreto 847/2011, Italy Decreto 21/3/73, FDA 21 CFR, Japan JHOSPA, and Chinese regulation GB9685-2008. The pigment does not comply with Japan JHPA.

Kronos 2211, 2220, and 2233 are representative of rutile pigments produced by a chloride process, representative of R2 compounds corresponding to DIN EN ISO 591 part 1, containing, respectively, a minimum 95.5, 92.5, and 96 wt. % TiO₂ and are stabilized, respectively, with compounds containing aluminum, aluminum with silicon, and aluminum with silicon. The scattering power of a plastisol formulation containing the same may be, respectively, approximately 105, 99, and 104. Various grades of titanium dioxide may be employed depending on the target design needs. For example, Kronos 2233 is a titanium dioxide which resists degradation of the carrier polymer and maintains tinting effects even at high processing temperatures.

Printex FP is representative of a pigment black 7 (color index #77266) compliant with 21 CFR § 178.3297.

PV Fast Green GNX is representative of a pigment green 7 (copper phthalocyanine) which is FDA compliant under 21 CFR § 178.3297 without limitation.

PV Fast Yellow HG is representative of a pigment yellow 180 (benzimidazolone) which is FDA compliant under 21 CFR § 176.170 for applications wherein the food contacting surface meets conditions of use B, C, D, E, F, G from Table 2. The pigment has not been declared compliant for applications meeting conditions of use A or H.

Irgazin Yellow K 2070 is representative of a pigment yellow 110 (isoindolinone) and complies with or otherwise is permitted by the following rules and regulations: EU regulation no 1935/2004/EC—Art. 3, EU (EC) Regulation 10/2011, AP(89)1, Germany BfR IX, Spain Real Decreto 847/2011, Italy Decreto 21/3/73, Australian regulation AS 2070-1999, and Chinese regulation GB9685-2008, being conditionally compliant with use restrictions under France Brochure 1227, FDA 21 CFR, and Japan JHOSPA and JHPA.

Bayferrox 3910 is representative of a pigment yellow 42 (iron oxide yellow: FeO(OH)·xH₂O). Not more than 3 ppm arsenic, 1 ppm cadmium, 10 ppm lead, or 1 ppm mercury is lost on drying of the pigment, and the pigment complies with or otherwise is permitted by the following rules and regulations: EU AP(89)1, Germany BIR IX, France Circulaire 176 dated 2 Dec. 1959, Netherlands Warenwet/Regeling Verpakkingen; Uitvoeringsvoorschriften CIII-55, Spain Resolucion date 4.1L1982 in accordance with Art. 5 of Royal Decree 211/1992, Australian AS 2070.6, USA 21 CFR 178.3297, and Japan JHOSPA.

Irgazin Red K 3840 is representative of a pigment red 254 (diketopyrrolopyrrole) and complies with or otherwise is permitted by the following rules and regulations: EU regulation no 1935/2004/EC—Art. 3, EU (EC) Regulation 10/2011, AP(89)1, Germany BIR IX, France Brochure 1227, Spain Real Decreto 847/2011, Italy Decreto 21/3/73, and Australian regulation AS 2070-1999, being conditionally compliant with use restrictions under FDA 21 CFR, Japan JHOSPA and JHPA, and Chinese regulation GB9685-2008.

Cromophtal Orange GP is representative of a pigment orange 64 (disazo condensation) and complies with or otherwise is permitted by the following rules and regulations: EU regulation no 1935/2004/EC—Art. 3, EU (EC) Regulation 10/2011, AP(89)1, Germany BIR IX, France Brochure 1227, Spain Real Decreto 847/2011, Italy Decreto 21/3/73, and Australian regulation AS 2070-1999, being conditionally compliant with use restrictions under FDA 21 CFR, Japan JHOSPA, and Chinese regulation GB9685-2008.

Heliogen Blue K 7090 is representative of a pigment blue 15:3 or unchlorinated copper phthalocyanine (beta form with approx. . . . 11 wt. % copper) and complies with or otherwise is permitted by the following rules and regulations: EU regulation no 1935/2004/EC—Art. 3, AP(89)1, Germany BfR IX, Japan JHPA, and Australian regulation AS 2070-1999, being conditionally compliant with use restrictions under EU (EC) Regulation 10/2011, France Brochure 1227, Spain Real Decreto 847/2011, Italy Decreto 21/3/73, FDA 21 CFR, Japan JHOSPA, and Chinese regulation GB9685-2008. Unavoidable impurities are suppressed to less than 20 ppm antimony, less than 20 ppm arsenic, less than 20 ppm lead, less than 30 ppm cadmium, less than 50 ppm chromium, less than 20 ppm selenium, less than 20 ppm mercury, and less than 20 ppm zinc. Any primary aromatic amines are also suppressed to less than 100 ppm.

Heliogen Green K 8730 is representative of pigment green 7 or a chlorinated copper phthalocyanine (with approx. 5.6 wt. % copper) and complies with or otherwise is permitted by the following rules and regulations: AP(89)1, being conditionally compliant with use restrictions under EU regulation no 1935/2004/EC—Art. 3, EU (EC) Regulation 10/2011, Germany BfR IX, France Brochure 1227, Spain Real Decreto 847/2011, Italy Decreto 21/3/73, FDA 21 CFR, Japan JHOSPA, Japan JHPA, Australian regulation AS 2070-1999, and Chinese regulation GB9685-2008. Unavoidable impurities are suppressed to less than 20 ppm antimony, less than 20ppm arsenic, less than 20 ppm lead, less than 30 ppm cadmium, less than 50 ppm chromium, less than 20 ppm selenium, less than 20 ppm mercury, and less than 20 ppm zinc. Any primary aromatic amines are also suppressed to less than 100 ppm.

The compositions of the present disclosure can be compounded and formed into a polymer article using any technique known in the art. For instance, the respective composition can be intensively mixed to form a substantially homogeneous blend. The blend can be melt kneaded at an elevated temperature, such as a temperature that is higher than the melting point of the polymer utilized in the polymer composition but lower than the degradation temperature. Alternatively, the respective composition can be melted and mixed together in a conventional single or twin screw extruder. Preferably, the melt mixing is carried out at a temperature ranging from 100 to 280° C., such as from 120 to 260° C., such as from 140 to 240° C. or 180 to 220° C. After extrusion, the compositions may be formed into pellets. The pellets can be molded into polymer articles by techniques known in the art such as injection molding, thermoforming, blow molding, rotational molding and the like.

The production of pellets, for example, may provide a raw material for the production of medical devices. In one embodiment, the pellets are certified according to at least one biocompatibility or food contact standard. In one embodiment, careful attention to the formulation and processing of the pellets may permit the biocompatibility or food contact certification to convey from the raw material to the finished product, reducing the regulatory burden on the production process.

Polymers as described herein may be produced in accordance with the current standards for Good Manufacturing Practice (GMP). For example, as required by EU Reg. No. 2230/2006, GMP includes (1) a quality assurance system ensuring that produced articles comply with rules applicable to them, such as by selecting starting materials which comply with pre-established specifications that ensure compliance of the article; (2) a quality control system monitoring the application of GMP and promptly correcting any failures to comply with GMP; and (3) a documentation system cataloging specifications and manufacturing formulae relevant to the compliance with standards pertaining to the manufactured article or the relevant GMP guidelines, made available to competent authorities upon request.

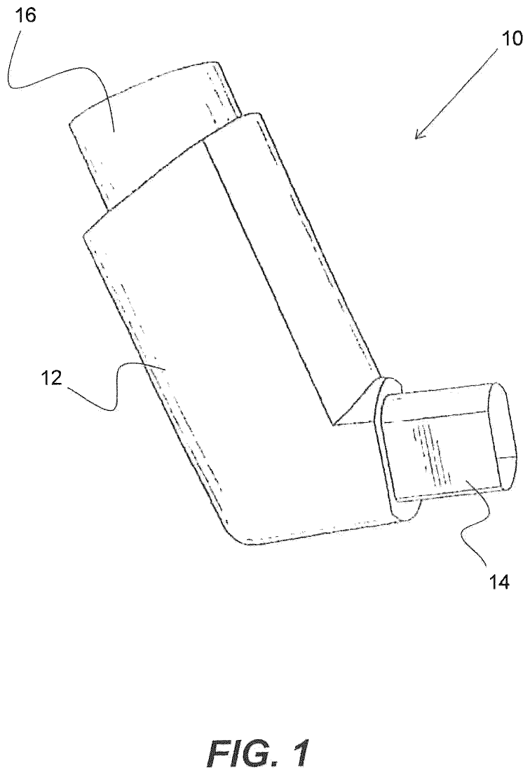

Exemplary embodiments include various medical devices. For instance, referring to FIG. 1, an inhaler 10 is shown. The inhaler 10 includes a housing 12 attached to a mouthpiece 14. In operative association with the housing 12 is a plunger 16 for receiving a canister containing a composition to be inhaled. The composition may comprise a spray or a powder. During use, the inhaler 10 administers metered doses of a medication, such as an asthma medication to a patient. The asthma medication may be suspended or dissolved in a propellant or may be contained in a powder. When a patient actuates the inhaler to breathe in the medication, a valve opens allowing the medication to exit the mouthpiece. In accordance with the present disclosure, the housing 12, the mouthpiece 14 and the plunger 16 can all be made from a polymer composition as described above.

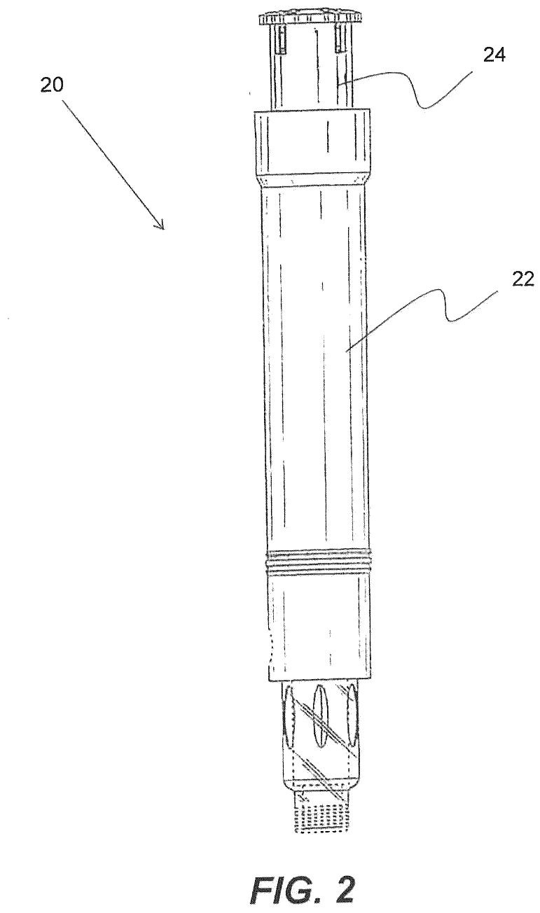

Referring to FIG. 2, another medical product that may be made in accordance with the present disclosure is shown. In FIG. 2, a medical injector 20 is illustrated. The medical injector 20 includes a housing 22 in operative association with a plunger 24. The housing 22 may slide relative to the plunger 24. The medical injector 20 may be spring loaded. The medical injector is for injecting a drug into a patient typically into the thigh or the buttocks. The medical injector can be needleless or may contain a needle. When containing a needle, the needle tip is typically shielded within the housing prior to injection. Needleless injectors, on the other hand, can contain a cylinder of pressurized gas that propels a medication through the skin without the use of a needle. In accordance with the present disclosure, the housing 22 and/or the plunger 24 can be made from a polymer composition as described above.

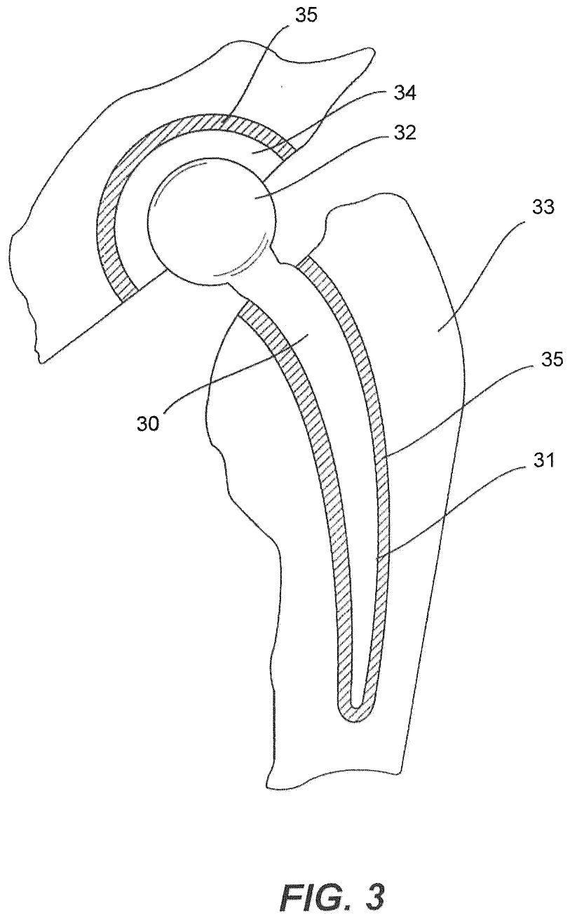

Referring to FIG. 3, in another example, a hip implant is illustrated. The hip implant includes a hip prosthesis 30 having a stem 31 and a joint engaging member or head 32. The hip prosthesis 30 can be made from a polymer material in accordance with the present disclosure. As shown, the hip prosthesis 30 has been inserted into a cavity reamed into a bone 33, such as a femur.

The hip implant illustrated in FIG. 3 further includes an acetabular cup 34 that can also be made in accordance with the present disclosure. Acetabular cup 34 comprises a joint engaging member that is adapted to receive the head 32 of the hip prosthesis 30. In order to implant the hip prosthesis 30 and the acetabular cup 34, both articles can be cemented to the bone using a bone cement 35.

In addition to being used to produce hip implants, the polymer composition of the present disclosure can also produce various other orthopedic devices. For instance, the polymer composition is well suited to producing a knee prosthesis, including a tibia plateau.

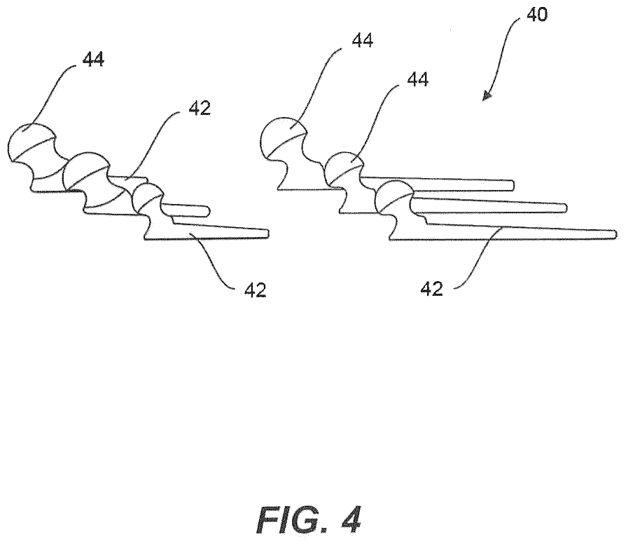

In one embodiment, the polymer composition is used to produce trial sizers that assist a physician or surgeon with determining the correct size of an implant during an operation. In one embodiment, each of the trial sizers may be color coded in order to indicate a particular size. For instance, referring to FIG. 4, a plurality of orthopedic devices 40 are shown. The plurality of orthopedic devices 40 can all be made from the polymer composition of the present disclosure. As shown, each orthopedic device 40 includes a stem 42 and a joint engaging member or head 44. The entire orthopedic device can be made from the polymer composition or, alternatively, only the head or joint engaging member may be made from the polymer composition of the present disclosure. In yet another embodiment, the polymer composition may comprise a coating that is used to produce the joint engaging member.

As shown in FIG. 4, each orthopedic device 40 has a different dimension or size and, as described above, can each be produced with a different color. During a surgical operation, a bone site can be prepared for insertion of the implant. The plurality of orthopedic devices 40 as shown in FIG. 4 can then be used by the surgeon to determine the proper size of the prosthesis that should be used on the particular patient. Once the appropriate size is determined, the surgeon can select an orthopedic device from a second plurality of devices that is to be inserted into the body of the patient. The second plurality of orthopedic devices can be made from the same polymer composition or can be made from a different material.

Polymers prepared according to the present disclosure may be better understood in light of the following exemplary embodiments.

### EXAMPLES

A polyoxymethylene polymer was prepared according to the present disclosure by mixing:
95.28 parts POM flake,
2.5 parts Iriotec 8208,
1 part Titandioxid Kronos 2211,
0.5 part POM Terpolymer,
0.4 part Irganox 245 FF,
0.2 part EBS wax (vegetable),
0.1 part tricalcium citrate, and
0.02 part dicyandiamide.

An extractable formaldehyde test resulted in 4.2 μg/cm².

A second example polyoxymethylene polymer was prepared by mixing:
9682 parts POM Pulver 13034,
318 parts PB MT RM,
64 parts Titandioxid Kronos 2233,
2.7 parts PV Fast Green GNX,
1.6 part Heliogen Blue K 7090,
1.6 part Heliogen Green K 8730, and
0.1 part Bayferrox 3910.

An extractable formaldehyde test resulted in 1.3 μg/cm². The above polymer composition is laser markable.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged either in whole or in part. Furthermore, those of ordinary skill in the art will appreciate that the foregoing description is by way of example only, and is not intended to limit the invention so further described in such appended claims.

What is claimed:

1. A polymer composition comprising:
a polyoxymethylene polymer having a melt volume flow rate of less than about 30 cm³/10 min when tested at 190° C. under a load of 2.16 kg, the polyoxymethylene polymer being present in the polymer composition in an amount of at least 80% by weight;
a thermoplastic elastomer present in the composition in an amount greater than about 5% by weight and in an amount less than about 15% by weight;
an acid scavenger present in the composition in an amount less than about 1% by weight;
a lubricant present in the composition in an amount greater than about 0.075% by weight and in an amount less than about 0.5% by weight;
a copolyamide present in the composition in an amount greater than about 0.0001% by weight and in an amount less than about 0.3% by weight; and
an antioxidant present in the composition in an amount greater than about 0.075% by weight and in an amount of less than about 1% by weight.

2. A polymer composition as defined in claim 1, wherein the polyoxymethylene polymer contains net chloroform-soluble extractives of less than 0.5 mg/in² in accordance with 21 CFR § 177.2470 to 2480 wherein the extractives are prepared according to 21 CFR § 1.75.300(d).

3. A polymer composition as defined in claim 1, wherein the polymer composition produces less than 0.5 mg/in² of extractable formaldehyde when tested according to 21 CFR § 177.2470 to § 177.2480 using chloroform.

4. A polymer composition as defined in claim 1, wherein the composition passes European regulation EC 10/2011 testing.

5. A polymer composition as defined in claim 1, wherein the composition contains the polyoxymethylene polymer in an amount greater than about 85% by weight.

6. A polymer composition as defined in claim 1, wherein the acid scavenger comprises tricalcium citrate.

7. A polymer composition as defined in claim 6, wherein the tricalcium citrate is present in the polymer composition in an amount from about 0.05% by weight.

8. A polymer composition as defined in claim 1, wherein the antioxidant comprising a sterically hindered phenol and is present in the composition in an amount of from about 0.075% by weight to about 0.5% by weight.

9. A polymer composition as defined in claim 1, wherein the polymer composition further contains a nucleant, the nucleant comprising a terpolymer.

10. A polymer composition as defined in claim 1, wherein the lubricant comprises ethylene bis(stearamide).

11. A polymer composition as defined in claim 10, wherein the ethylene bis(stearamide) is present in the composition in an amount of from about 0.2% by weight to about 0.5% by weight.

12. A polymer composition as defined in claim 1, wherein the thermoplastic elastomer comprises a thermoplastic polyurethane elastomer.

13. A polymer composition as defined in claim 12, wherein the thermoplastic elastomer is present in the composition in an amount of from about 8% by weight to about 10% by weight.

14. A polymer composition as defined in claim 1, wherein the composition is in the form of compounded pellets.

15. A polymer composition as defined in claim 1, wherein the copolyamide is present in the composition in an amount of from about 0.0001% by weight to about 0.1% by weight.

16. A polymer composition as defined in claim 1, wherein the polyoxymethylene polymer contains terminal hydroxyl groups and side groups in an amount of greater than about 18 mmol/kg.

17. A polymer composition as defined in claim 1, wherein the composition does not contain any reinforcing fibers.

18. A molded article made from the polymer composition as defined in claim 1.

19. A molded article in claim 18, wherein the molded article comprises an inhaler or a medical injector.
